(19) United States
(12) Patent Application Publication
(10) Pub. No.: US 2017/0127966 A1
(43) Pub. Date: May 11, 2017

(54) ELECTRODE DEVICE FOR WEARABLE OR HANDHELD APPARATUS

(71) Applicant: Hong Yue Technology Corporation, Hsinchu City (TW)

(72) Inventors: Chi-Sheng Wu, Hsinchu City (TW); Yu-Sheng Liao, Hsinchu City (TW); Mei-Hua Liao, Hsinchu City (TW)

(73) Assignee: Hong Yue Technology Corporation, Hsinchu City (TW)

(21) Appl. No.: 15/291,081

(22) Filed: Oct. 12, 2016

(30) Foreign Application Priority Data
Nov. 10, 2015 (TW) 104137091

(51) Int. Cl. A61B 5/0408; A61B 5/00; A61B 5/0402

(52) U.S. Cl. CPC A61B 5/0408; A61B 5/0402; A61B 5/0002; A61B 5/681; A61B 2562/0209

(57) ABSTRACT

An electrode device has a conductive body, a silver layer situated above the conductive body, a silver compound layer situated above the silver layer, a liquid absorbing and releasing element situated above the silver compound layer, a shielding element configured to cover an upper surface and a portion of a side surface of the liquid absorbing and releasing element, and a moving mechanism configured to move the shielding element reciprocally between at least two positions.

### BACKGROUND OF THE INVENTION

Field of the Disclosure

The present invention relates to an electrode device for a wearable or handheld apparatus, and more particularly to the electrode device for a wearable or handheld apparatus applied for recording electrophysiological signals.

Description of Related Art

Since 1900, heart disease has been the leading cause of death in the United States. Through the electrocardiogram (ECG), an early diagnosis of heart disease can be given. However, for patients suffering an early-stage heart disease, the electrocardiogram results are not always abnormal. It is in fact only occasionally that the ECG shows abnormalities. The occasional abnormal results represent the symptoms that a patient experiences once or twice in a day for a few seconds such as chest pain or stifled breathing. Aside from the occasional abnormal results, the ECG displays normal results for the patient for the rest of the time. Abnormal ECG results are the basis for the diagnosis of heart disease.

The Holter ECG recorder is best when it comes to recording occasional abnormalities, as it can continuously record for 24 hours, approximately 100,000 heartbeats recorded from chest and limb lead electrocardiograms that may later be handed to a cardiologist for careful inspection. However, use of the Holter ECG recorder requires many attachments, i.e., physiological electrode pads, to the limbs and chest, which may be uncomfortable for the wearer. At present, the only approved standard physiological electrode for clinical application is a structure comprised of silver/silver chloride/potassium chloride (Ag/AgCl/KCl, refer to ANSI/AAMI EC12: Disposable ECG electrodes), which is also widely used in electrochemical and electrophysiological research.

The physiological electrode contains silver, a silver chloride membrane formed by oxidizing the surface of the silver sheet, and a layer of potassium chloride enriched gel. When the physiological electrode is attached onto a patient's skin the potassium ion and chloride ion are used to conduct electrophysiological signals generated by the heart, muscles or brain cells activities through large blood vessels to small blood vessels (containing blood which is good conductor) and conduct the electrophysiological signals through sweat glands (sweat contains potassium ion, sodium ion, and chloride ion so that it conducts electricity easily) of a patient's skin to the Ag/AgCl/KCl interface. In the two electrochemical reactions displayed below, the electrocardiogram device converts the electrophysiological signal expressed by ion flows (Cl⁻, Na⁺, HCO⁻, K⁺, etc.) in the blood into an electron stream in a metal conductor (silver, copper, etc.) or an electron/hole stream in semiconductor devices. Even though the electron stream or the hole stream flows in different conductors (silver, copper, or silicon), the noise caused by the interface is still very small and negligible, so that processes such as amplification and filtering can be handled in an integrated circuit easily.

Ag←→Ag⁺+e−;

Ag⁺+Cl^{-←→AgCl;}

Potassium chloride instead of sodium chloride is used in the gel because the diffusion rates of chloride ion and potassium ion in water are very close, and the membrane potential caused by diffusing through a semi-permeable member is very small. On the other hand, the diffusion rates of chloride ion and sodium ion in water are very different, and additional membrane potential may be formed easily, and therefore interferes with the physiological signals and provides a relatively inaccurate result. However, the potassium ion concentration in an intracellular fluid is relatively higher, and the potassium ion concentration in extracellular fluid, blood, and sweat is too low to maintain a stable electric potential. Therefore, because the electrical conductivity built only by the sweat on epidermis is not good enough, it is necessary to add a potassium chloride solution. In a physiological electrode in compliance with the ANSI/AAMI EC12 standard, the concentrations of both silver ion and chloride ion in the gel are maintained stable, such that as long as the temperature of the electrode is close to body temperature, the half-cell potential formed by the aforementioned electrochemical reaction will be stable and the electrophysiological signal can be obtained successfully. The electrophysiological signal is conducted through the electrode attached onto the user's skin to an analog circuit and then to a digital circuit, and finally processed by firmware and software to produce an electrocardiogram (ECG), an electromyogram (EMG), or an electroencephalogram (EEG) which is provided for diagnosing different kinds of diseases and maintaining human health.

In practical applications, silver itself is more expensive and softer, and thus a harder copper alloy instead of silver is used for making the main body of an electrode, and a side of the main body is plated with a silver membrane, and then the silver membrane is submerged into dilute hydrochloric acid and positive electricity is passed to perform anodic process, and the silver surface is oxidized to form a silver chloride membrane. The other side of the copper body is plated with tin, nickel or gold to prevent the copper body from being oxidized in air, so that the copper body can be touched or soldered with other conductors to connect to a semiconductor circuit. At present, the physiological electrodes commonly used in hospitals are in the shape of a circular disc with a core which is a silver/silver chloride plate with a diameter of approximately 10mm, a layer of gel on one side for contacting a user's skin, a snap-in button on the other side to couple an electric wire extended from an instrument such as an electrocardiograph, an electromyograph, or an electroencephalograph, etc.

Please refer to FIG. 1 for three limb leads, Lead I, II, and III, defined by the Einthoven triangle, which is the most widely used electrocardiograms for disease diagnosis. From the definition, the electrodes may be placed at any position of the user's upper or lower limbs to obtain the limb leads. However, in a practical application, the electrodes are usually attached to the interior side of the wrist or ankle, because in such areas there are only thin tendons and almost no muscle in between the electrodes and major blood vessels which prevents motion artifact to limb leads caused by the movement of muscles. For example, respiration is inevitable and bound to result in the baseline wandering of Lead I. As to the diagnostic electrocardiograms for cardiologist reviewing, high quality and low motion artifact are required, hence the patient must lie still and have electrodes attached to the interior side of the wrists and ankle to avoid motion artifact. As for handheld or wearable apparatuses, more severe motion artifact is encountered because such apparatuses are often used to capture electrophysiological signals from a human body in action rather than from a still human body.

Although the silver/silver chloride/potassium chloride (Ag/AgCl/KCl) structured electrode can be used to obtain high quality electrocardiograms, contact with silver, chlorine, potassium ion and gel for a long time will cause uncomfortable swelling and itching to the skin, and purulent inflammation and fester are common in intensive care units after such an electrode has been used continuously for more than two days. Furthermore, the electrical wires which are tied onto a patient's body from an electrocardiograph interfere the patient's daily life activities. Most patients with heart disease are unwilling to continue to wear the Holter electrocardiograph after wearing it for one day.

To overcome this problem, handheld or wearable Event ECG recorders are available in the market for obtaining electrocardiograms for whenever a patient does not feel very well; otherwise the event electrocardiograph recorder does not touch the patient's skin nor capture electrocardiograph signals. Since the gel is sticky, it causes discomfort and may interfere with daily life activities. Furthermore, the silver chloride is dark brown color which is unattractive and may be damaged or peeled off easily. Therefore, most handheld or wearable event ECG recorders do not adopt the standard silver/silver chloride/potassium chloride (Ag/AgCl/KCl) physiological electrodes nor the gel, but use metal or electrically conductive compounds as conductors to connect directly to the human body in order to capture electrophysiological signals. Such electrodes are generally called dry electrodes. According to the study conducted by Michael Neuman et al (refer to Neuman, M. R., Chap. 48, “Biopotential Electrodes.” in “The Biomedical Engineering Handbook: Second Edition”, Edited by Joseph D. Bronzino), dry electrodes has the drawbacks of higher impedance, and unstable interface potential easily interfered by temperature change, producing a low frequency drift or a high frequency (60 Hz or radio frequency) noise (when compared with the standard silver/silver chloride/potassium chloride physiological electrode). Therefore, the quality of the electrophysiological signals obtained by the dry electrode is worse than that obtained by the standard physiological electrode. This is because the dry electrode does not have a stable electrochemical reaction to maintain the interface potential, nor has any gel to preserve the potassium chloride solution; instead, it relies on the moisture (or sweat) on the skin to conduct the electrophysiological signals to the electronic circuit. There is a far greater amount of sodium ions than potassium ions in sweat, and sodium diffusion causes membrane potential to build low frequency drift interference. Furthermore, in a dry and cold environment, the moisture or sweat on skin is so scarce that it provides little electrical conductivity, hence the motion artifact maybe very significant.

FIG. 2 shows an ideal limb lead I electrocardiogram, where P, T, and U waves are low frequency and small amplitude signals, while only the QRS wave group has a relatively greater amplitude. Therefore, the electrocardiogram is the most vulnerable to low frequency drift interference among the three electrophysiological signals, electrocardiogram, electromyogram, and electroencephalogram. Dry electrodes always give low frequency drifts or high frequency (50, 60 Hz or radio frequency) noises, so that the waves P, T, and U are muffled by the noise (refer to FIG. 2 of US 2014/0249398 A1), and only the QRS complex with a greater amplitude can be identified. This type of electrocardiogram which is only capable of identifying QRS complex, is suitable for measuring heart rate or analyzing heart rate variability (HRV), but is not competent for the diagnosis of heart disease.

In addition, the dry electrode has a substantial drawback for the application of the event ECG recorder. It responds so slowly that it cannot obtain ECG signals until it is in contact with the skin for more than ten seconds, which is not helpful to the early-stage heart disease patients whose abnormal electrocardiogram lasts for only several seconds. The reasons that dry electrodes respond slowly are described below.

Please refer to FIG. 3 for a schematic circuit diagram showing a physiological signal being conducted through the skin and the electrode to an electrophysiological signal amplifier circuit. An input terminal of the conventional electrophysiological signal amplifier circuit is an instrumentation amplifier (refer to the AD8220 datasheet published by Analog Devices Inc.), some resistors, capacitors and diodes must be connected together with the input terminal to protect the instrumentation amplifier from being damaged by electrostatic discharge (ESD) from human body and to reduce radio frequency interference. In addition, the output terminal of the instrumentation amplifier is also connected with high-pass and low-pass filters comprised of resistors and capacitors. Before the electrode is in contact with human body, electric charges of these capacitors and diodes are likely to drive the output of the instrumentation amplifier to the upper or lower saturation region, making it unable to present the electrophysiological signal. After the electrode is in contact with the human body, these capacitors and diodes start to be charged or discharged by the human body thus that the circuit reaches a stable state, and then the output of the instrumentation amplifier goes to its linear region so that the electrophysiological signal can be presented. The time from the electrode being in contact with the human body to the instrumentation amplifier being able to present the electrophysiological signal is defined as transient time. When a standard physiological electrode (in compliance with AAMI EC12) is used, its resistance should be smaller than 3000 Ohms (4.2.2.1, AAMI EC12), which results in the transient time being usually much shorter than one second. In such cases the users can hardly feel it. When dry electrodes are used in a dry and cold environment, the resistance may be up to millions of ohms, which may result in the transient time taking up to more than ten seconds. For a patient lying in a physician's office for three minutes to acquire complete 12-lead electrocardiogram by standard physiological electrodes, the transient time of more than ten seconds does not affect the results. However, the transient time is critical for determining whether or not the electrodes are useful to patients who use a wearable or handheld apparatus to acquire electrocardiogram with an occasional abnormality lasting for only several seconds.

In summary, the quality of electrocardiograms acquired by dry electrodes is not ideal; these electrocardiograms can be used only for computing heart rate or heart rate variability (HRV) analyzing, but are incapable of regular heart disease diagnosis. Dry electrodes currently have drawbacks when applied to a handheld or wearable an event ECG recorder. As disclosed in U.S. Pat. Nos. 5,289,824 and 5,613,495, a wrist watch type event ECG with trade name “Hearth Watch III” is cleared to market by the United States Food and Drug Administration (USFDA) with a clearance number K945476. In this product, one electrode is disposed on the inner side of the watch body for contacting the wrist that wears the watch for a long time. While recording, a wearer may gently touch the electrode at the upper surface of the watch with a finger of the other hand not wearing the watch, so that each of both hands touches an electrode to obtain the R-peaks of the electrocardiogram to compute heart rate. However, the electrodes of Hearth Watch III are made of titanium that will not be rusted easily or the electrodes are covered by an electrically conductive titanium nitride, titanium carbide or carbon titanium nitride to make the surface of the electrode more aesthetic. There is no silver/silver chloride sheet or potassium chloride solution in the electrode; thus, if the skin of the finger is so thick or dry that its electrical impedance is very high, the user will need to wait for a longer time to obtain electrocardiograms. In addition, because skin on the finger has greater electrical impedance, the electrocardiograph signal is easy to be interfered by 60 Hz, radio wave, muscle contraction, and motion artifact.

U.S. Pat. No. 5,778,882 discloses a handheld health information tracking device, but does not mention the structure of its electrode.

U.S. Pat. No. 6,587,712 B1 discloses a handheld electrocardiograph signal monitoring device comprising a probe (including two electrodes) similar to a handheld stethoscope held by the user. When the user presses the probe onto one's chest, an electrocardiograph signal is obtained. However, the obtained electrocardiogram signal is not a limb lead electrocardiogram defined by Einthoven, and this patent does not disclose the material for making the electrode.

U.S. Pat. No. 7,171,259 B2 states the shortcomings of the aforementioned wrist watch type heart rate measuring device as follows. The user touches the outer side of the wrist watch with only a fingertip, so that the relative positions of both hands are unstable, finger muscle contraction causes interference, the contact area is too small, and the skin is too dry. As a result, the obtained electrocardiograph signal is very unstable and difficult to interpret. This patent proposes an improved structure comprising multiple electrodes situated on the outer side of a wrist watch for the user to fasten the wrist watch, e.g., to use the forefinger and thumb to make a loop to hold the watch, so that the relative position of both hands will be more stable, and the contact area will be greater, so as to obtain an electrocardiograph signal with preferred quality. This patent also proposes a transparent conductive material such as Indium-Tin-Oxide (ITO) plated onto the surface of the wrist watch, so that the surface also has the function of an electrode and obtains a greater contact area. However, this patent has not proposed the Ag/AgCl/KCl electrode structure.

U.S. Pat. No. 7,197,351 B2 discloses a handheld apparatus having two electrodes installed on a casing, wherein one electrode is used to contact the user while holding the casing with the right hand and the other electrode installed at the bottom of the apparatus contacts the user's left chest in order to obtain a limb lead I electrocardiogram. This patent does not disclose the material for making the electrode. The user's left chest must be exposed during use, and the right-hand electrode is electrically connected to large blood vessels of the wrist through the muscles of the palm and fingers, so that the right hand must apply an appropriate force to hold the apparatus and contact the left chest during use, it is therefore inevitable to have motion artifact caused by the muscle action of the right hand.

U.S. Pat. No. 7,471,976 B2 discloses a business card type handheld apparatus, with gel-free electrodes installed on both sides of the apparatus separately to contact user's fingers to obtain an electrocardiogram. This patent just discloses that the electrode is made of a conductive metal or rubber, and has not used a potassium chloride solution.

U.S. Pat. No. 7,894,888 B2 discloses a wrist watch type electrocardiogram signal recorder having an electrode installed at the inner surface of its main body, and two electrodes are installed at the outer surface of its main body and a watchband. While the user's finger and left lower abdomen touch this wrist watch, the three limb leads electrocardiogram defined by Einthoven can be captured. This patent proposes to use a pressure, infrared or electrical impedance sensor to find out whether or not the electrodes are in contact with the human body to timely start an electrocardiogram circuit, but does not disclose the material for making the electrodes.

U.S. Pat. No. 7,896,811 B2 discloses a handheld apparatus (such as a mobile phone) having two electrodes and two pressure sensors installed to the casing of the apparatus, but this patent has not disclose the material for making the electrode or the use of potassium chloride solution.

U.S. Pat. No. 8,082,762 B2 discloses a conductive material and an electrode having an elastic textile, while the electrode is installed in clothing. In one embodiment, a silver plated copper wire and a silver metalized nylon yarn are used for conducting electricity, but not as a silver/silver chloride structure, which makes it unable to maintain a stable electrochemical potential.

U.S. Pat. No. 8,214,008 B2 discloses the use of a conductive material and an elastic textile as an electrode which is installed into clothing. In one embodiment, a fiber containing silver particles is used for conducting electricity instead of silver/silver chloride structure, which makes it unable to maintain a stable electrochemical potential.

U.S. Pat. Nos. 8,244,336 B2 and 8,095,199 B2 discloses a handheld apparatus having two electrodes installed to the casing, with one of the electrodes touching a user when the user's right hand holds the apparatus, and the other electrode situated at the bottom of the apparatus touches the user's left chest to obtain a limb lead electrocardiogram of the Limb Lead I. This patent has not listed the material for making the electrode. The user's left chest must be exposed during use, and the right-hand electrode connects to large blood vessels of the wrist through the muscles of the palm and finger, so that the right hand must apply an appropriate force to hold the apparatus and touch the left chest during use, it is therefore inevitable to have a motion artifact caused by the muscle movement of the right hand.

U.S. Pat. No. 8,359,088 B2 discloses a case type electrocardiogram monitor for folder-type cellular phones, with a pair of electrodes made of stainless steel, brass, or copper for capturing an electrocardiogram.

U.S. Pat. No. 2009/0048526 A1 also discloses a wrist watch type electrocardiogram signal recorder that uses one wrist instead of both wrists to obtain electrical pulse signals to detect heartbeats. Such a recorder can obtain heart rate and compute heart rate variability, but the recorder cannot obtain a limb lead electrocardiogram defined by Einthoven. The physiological or pathological information provided by the recorder is far inferior to that provided by the limb lead electrocardiogram.

U.S. Pat. No. 2011/0066042 A1 discloses a handheld apparatus comprising an electrocardiogram, heart sound, and acceleration sensing circuit. This patent does not disclose the material for making the electrode.

U.S. Pat. No. 2013/0261414 A1 discloses a handheld apparatus comprising two electrodes and two photo plethysmograph (PPG) sensors, with the electrocardiogram and the pulse signal being obtained by the user's two forefingers. However, it does not disclose the materials for making the electrodes and the use of the potassium chloride solution.

U.S. Pat. No. 2014/0249398 A1 discloses a method of computing pulse transient time (PTT) by using a handheld apparatus (such as a mobile phone). The handheld apparatus has two electrodes for obtaining electrocardiogram signals from both hands of a user, and a camera installed into the handheld apparatus obtains a pulse signal from the user's pupil, while the time difference of the two is used as the pulse transient time. However, this patent application does not disclose the material for making the electrodes.

The methods mentioned above adopt a dry electrode and thus have the drawbacks of high impedance, baseline wandering, and long transient time. To overcome these drawbacks, a handheld apparatus in the size of a business card such as the product (trade name ecg@home with USFDA clearance number) patented by U.S. Pat. Nos. 5,928,141 and 6,345,196 B1 was implemented. The handheld apparatus has silver/silver chloride electrodes installed separately on both ends to be touched by a user's fingers, and the surface of the electrode has many pores for accommodating potassium chloride solution. When not in use, this product is stored in a bag or a pocket. When it is necessary to use this product, a potassium chloride solution is dropped on the electrodes to increase the electrical conductivity before starting the recording process. Such preparation takes approximately ten seconds. As for early heart disease patients that experience chest pain for several seconds, it is difficult to capture abnormal electrocardiograms.

In summation of the description above, there is a need to capture an occasional abnormal electrocardiogram timely for the diagnosis of diseases and design a method of obtaining the electrocardiogram without requiring a long time of attaching the electrodes and providing a handheld or wearable apparatus (such as a mobile phone or a wrist watch) with a silver/silver chloride/chloride structured electrode, and coating a potassium chloride solution onto an appropriate position of the user's body without any discomfort caused by the use of conductive gel. Preferably, there is no muscle between the skin in contact with the electrode and a large blood vessel, and the hand holding or wearing posture preferably allows the user to relax as much as possible and requires no application of force to reduce the motion artifact caused by muscle movement. After the recording takes place, the electrode is removed from the skin to avoid allergy and itching of the skin.

### SUMMARY OF THE INVENTION

Therefore, the primary objective of the present invention is to provide an electrode device for a wearable or handheld apparatus with the features of easy and timely application free of physiological burden, and accurate and reliable recording. In an aspect, the electrode device comprises: a conductive body, a silver layer situated above the conductive body, a silver compound layer situated above the silver layer, a liquid absorbing and releasing element situated above the silver compound layer, a shielding element for covering an upper surface and a portion of a side surface of the liquid absorbing and releasing element, and a moving mechanism configured to move the shielding element reciprocally between at least two positions.

In one embodiment of the present invention, the shielding element is for coupling a spring, engaging a fastener, or connecting a micro switch.

In one embodiment of the present invention, the shielding element is a curved thin structure made of an elastic flexible material.

In one embodiment of the present invention, the shielding element has an inwardly extended edge for gripping the liquid absorbing and releasing element.

In one embodiment of the present invention, the silver layer and the silver compound layer jointly have an inwardly extended edge configured to grip the liquid absorbing and releasing element.

Another objective of the present invention is to provide a wearable apparatus for electrocardiogram recording with the features of easy and timely application, free of physiological burden, and accurate and reliable recording. In an aspect, the wearable apparatus for electrocardiogram recording includes an inner side of the wearable apparatus, an outer side of the wearable apparatus opposite to the inner side of the wearable apparatus, a main body, a first electrode, and a second electrode. The main body includes an electrocardiograph amplifier circuit, a microcontroller, a memory, a battery and a control firmware. Each of the first and second electrodes includes a silver layer, a silver compound layer, a shielding element, a liquid absorbing and releasing element, and a moving mechanism. The first electrode is situated on the inner side of the wearable apparatus, which is located between the radial ulnar arteries of the user's wrist. The second electrode is situated disposed on the outer side of the wearable apparatus. The first electrode and the second electrode are respectively connected to two input terminals of the electrocardiograph amplifier circuit.

In one embodiment of the present invention, the wearable apparatus for electrocardiogram recording further comprises a third electrode device situated on the outer side of the wearable apparatus, and the third electrode device includes a silver layer, a silver compound layer, a shielding element, a liquid absorbing and releasing element, and a moving mechanism. The third electrode device is coupled to a third input terminal of the electrocardiograph amplifier circuit.

In one embodiment of the present invention, the wearable apparatus for electrocardiogram recording is a wrist watch.

A further objective of the present invention is to provide a handheld apparatus for electrocardiogram recording with the features of easy and timely application, free of physiological burden, and accurate and reliable recording. In an aspect, the handheld apparatus for electrocardiogram recording comprises an apparatus body having a first side and a second side opposite to the first side, a first electrode device and a second electrode device. The apparatus body includes an electrocardiograph amplifier circuit, a microcontroller, a wireless transmission circuit, a memory, a battery and a control firmware. The first electrode device is disposed on the first side of the apparatus body and includes a silver layer, a silver compound layer, and a liquid absorbing and releasing element. The second electrode device is coupled to the apparatus body by a rotating arm and a pivot. The first electrode device and the second electrode device are respectively and electrically coupled to two input terminals of the electrocardiograph amplifier circuit. When the handheld apparatus is not in use for recording, the second electrode device is configured to cover the apparatus on the first electrode to prevent the first electrode device from being exposed to the outside. When it is necessary to use the handheld apparatus, the second electrode is configured to be turned to the second side of the main body to expose the first electrode device and the second electrode device which are configured for a user to touch.

In one embodiment of the present invention, the handheld apparatus for electrocardiogram recording further comprises a permanent magnet or a spring configured to make the second electrode device abut against the first electrode device.

In one embodiment of the present invention, the handheld apparatus for electrocardiogram recording further comprises a third electrode device situated on a lateral side of the handheld apparatus and electrically coupled to the electrocardiograph amplifier circuit. The third electrode device includes a silver layer, a silver compound layer, a shielding element, a liquid absorbing and releasing element, and a moving mechanism.

A further objective of the present invention is to provide a wrist-worn apparatus for electrocardiogram recording with the features of easy and timely application, free of physiological burden, and accurate and reliable recording. In an aspect, the wrist-worn apparatus for electrocardiogram recording comprises a primary wristband and a secondary wristband, wherein the primary wristband and the secondary wristband are configured to be worn separately on the two wrists of a user. The primary wristband includes a main body, a first band, a first electrode device, a first conductive contact and two first connecting lines. The main body includes an electrocardiograph amplifier circuit, a microcontroller, a battery, a memory and a control firmware. The first electrode device is situated on an inner side of the first band, positioning it between the radial and ulnar arteries of the wrist. The first electrode device includes a first silver layer, a first silver compound layer, a first shielding element, a first liquid absorbing and releasing element, and a first moving mechanism. The first electrode device is coupled to a first input terminal of the electrocardiograph amplifier circuit by one of the two first connecting lines, and the first conductive contact is coupled to a second input terminal of the electrocardiograph amplifier circuit by the other one of the two first connecting lines. The secondary wristband includes a second band, a second conductive contact, a second electrode device and a second connecting line. The second electrode device is on an inner side of the second band, positioning it between the radial and ulnar arteries of the other wrist. The second electrode device includes a second silver layer, a second silver compound layer, a second shielding element, a second liquid absorbing and releasing element, and a second moving mechanism. The second conductive contact is coupled to the second electrode device by the second connecting line.

In one embodiment of the present invention, the primary wristband further includes a switch. The switch shows two different logic states to the microcontroller with one of the different logic states indicating the primary wristband and the secondary wristband being in contact while the other of the different logic states indicating the primary wristband and the secondary wristband being not in contact.

When the physiological electrode device of a wearable or handheld apparatus in accordance with the present invention is not in use for recording electrophysiological signals, the shielding element covers the electrode to prevent the electrode from causing allergic reactions or itching of the skin due to the electrode touching the user's body, while stopping the evaporation of the electrochemical solution. When the physiological electrode device of a wearable or handheld apparatus in accordance with the present invention is being used for recording electrophysiological signals, a user may brush away the shielding element quickly to expose the electrode and allow the electrode to touch the user's body while compressing the liquid absorbing and releasing element to release an aqueous electrochemical solution. Through this process, the electrochemical solution may be coated onto the electrode to form a good electrical conductor to achieve the effects of reducing the transient time significantly and obtaining low-noise high-quality electrophysiological signals quickly. Therefore, the physiological electrode for a wearable or handheld apparatus in accordance with the present invention can overcome the drawbacks of current methods and record the abnormal electrocardiograms of early-stage heart disease patients without causing irritation of the skin.

### BRIEF DESCRIPTION OF THE DRAWINGS

FIG. 1 shows three limb leads defined by Einthoven triangle;

FIG. 2 shows a perfect electrocardiogram of Limb Lead I;

FIG. 3 is a schematic circuit diagram of a physiological signal conducted to an electrophysiological signal amplifier circuit through human skin and an electrode;

FIG. 4A is a cross-sectional view of an electrode device in accordance with one embodiment of the present invention;

FIGS. 4B and 4C are top views of a moving mechanism and a shielding element in accordance with different embodiment of the present invention, and also respectively show the positions before and after the shielding element is moved;

FIGS. 4D and 4E are schematic views of modifications of the embodiment of 4B and 4C, and show the positions before and after the shielding element is moved;

FIG. 4F is a schematic view of a fastener in accordance with one embodiment of the present invention;

FIG. 5 shows the system structure of an electrode device in accordance with one embodiment of the present invention applied to a wearable or handheld electrocardiogram recorder;

FIG. 6 is a cross-sectional view of an electrode device in accordance with another embodiment of the present invention;

FIG. 7A is a top view showing inner and outer sides of a wrist watch in accordance with one embodiment of the present invention containing an electrode device of the present invention;

FIG. 7B shows a using status of the wrist watch as depicted in FIG. 7A while recording an electrocardiogram;

FIG. 8 is a schematic view showing an inner side of a wrist of a human body;

FIG. 9A is a top view showing inner and outer sides of a wrist watch in accordance with another embodiment of the present invention containing an electrode device of the present invention;

FIG. 9B shows the status of a wrist watch as depicted in FIG. 9A—worn on a user's left hand;

FIGS. 10-12 show using statuses of a wrist watch as depicted in FIG. 9A recording electrocardiograms of first, second and third leads respectively;

FIG. 13 is a side view of mobile phone in accordance with one embodiment of the present invention containing an electrode device of the present invention in the statuses of capturing and not capturing an electrophysiological signal respectively;

FIG. 14 shows a modification of one embodiment of FIG. 13; and

FIG. 15 shows a status of a two-wristband wearable apparatus in accordance with one embodiment of the present invention containing an electrode device.

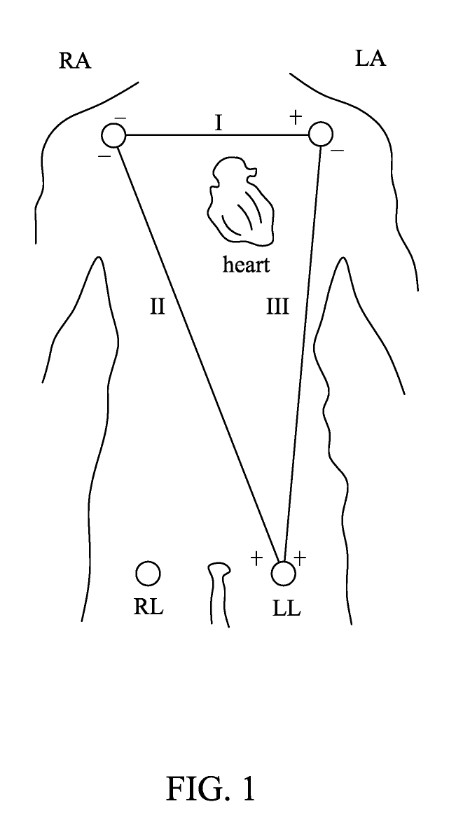

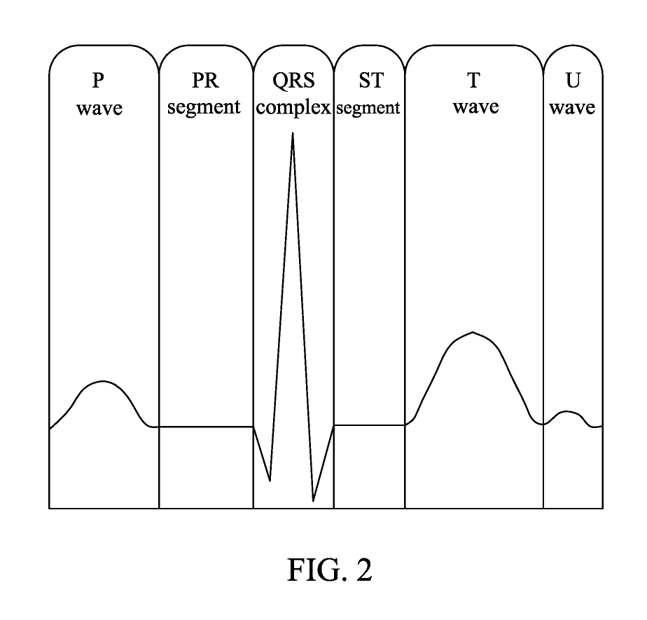

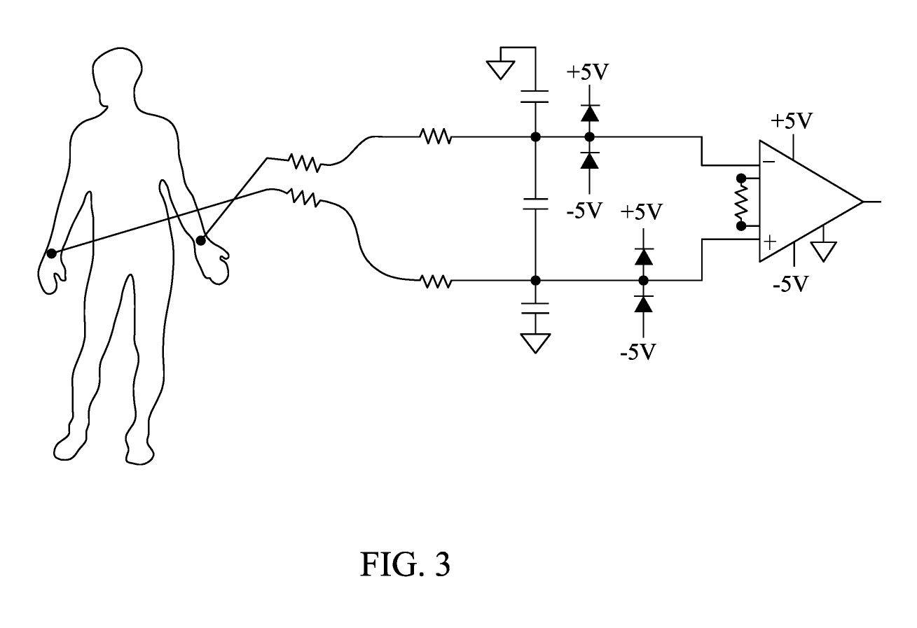

### DETAILED DESCRIPTION OF THE INVENTION

The technical characteristics, contents, advantages and effects of the present invention will be apparent with the detailed description of embodiments accompanied with related drawings as follows. The drawings are provided for the illustration, and the same numerals are used to represent respective elements in the embodiments. It is intended that the embodiments and drawings disclosed herein are to be considered illustrative rather than restrictive. The same numerals are used for representing the same respective elements in the drawings.

### First Embodiment

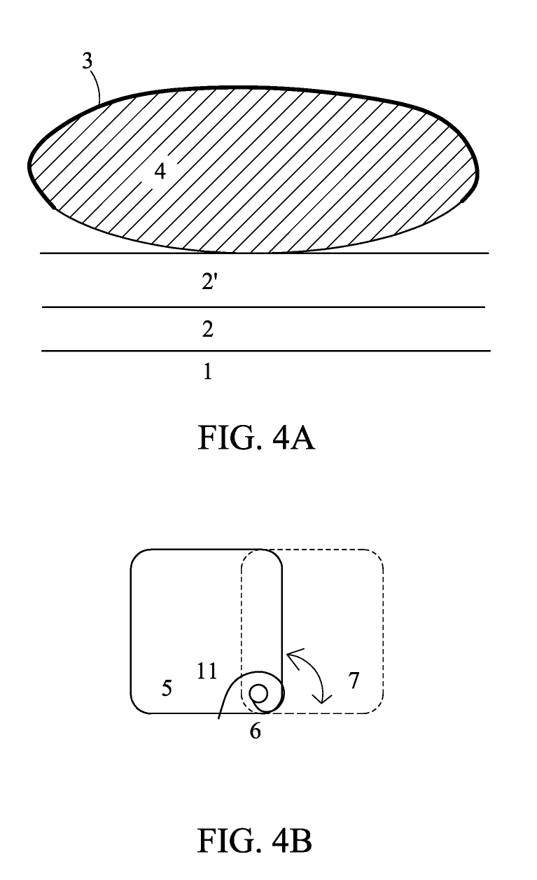

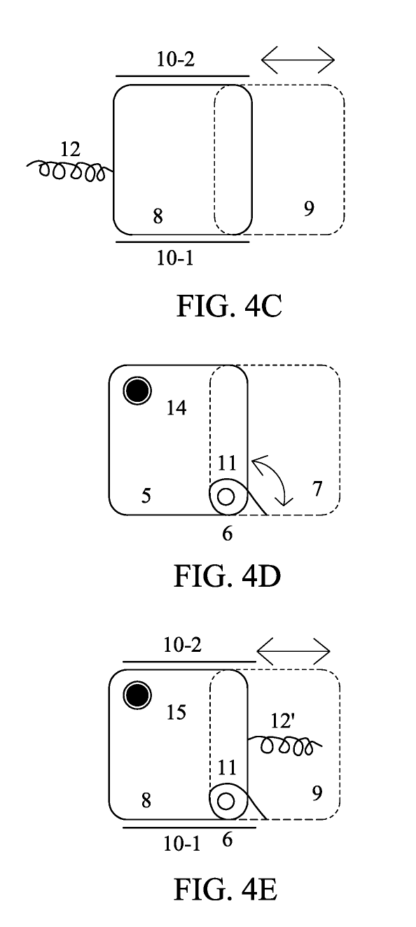

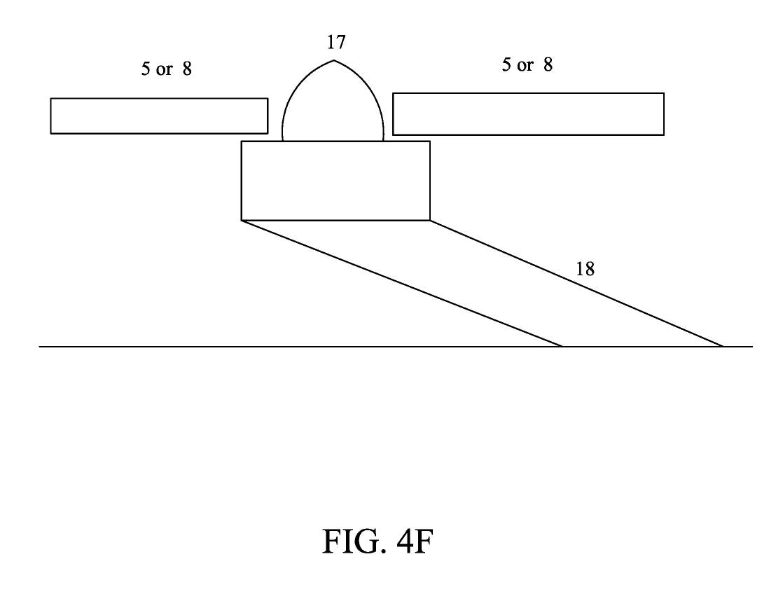

Please refer to FIG. 4A for a cross-sectional view of an electrode device in accordance with one embodiment of the present invention, the electrode device comprises a conductive body 1 such as a main body made of copper alloy, a silver layer 2 situated above the conductive body 1, a silver compound layer 2′ such as a silver chloride layer situated above the silver layer 2, a liquid absorbing and releasing element 4 situated above the silver compound layer 2′, a shielding element 3 such as a flap covering an upper surface and a portion of a side surface of the liquid absorbing and releasing element 4, and a moving mechanism (not shown in FIG. 4A, and described in details in FIGS. 4B˜4F) configured to move the shielding element 3 back and forth between at least two positions. As long as a good electrical conductivity is maintained, the conductive body 1 may be made of an alloy of any metal, a compound, an electrically conductive polymer, or any other material such as carbon nanotube. In one embodiment, the liquid absorbing and releasing element 4 has a compressible and porous structure such as an artificial sponge or a hydro-gel for absorbing an electrochemical solution such as a potassium chloride solution, and the volume of the liquid absorbing and releasing element 4 is slightly expanded after absorbing the electrochemical solution, so that a close contact with the upper surface of the silver compound layer 2′ and a close embedment in the shielding element 3 can be achieved to prevent it from falling off. It is noteworthy that both electrochemical solution and silver compound layer 2′ have a common ion, and the common ion of this embodiment is chloride ion. Of course, the common ion may be an ion other than the chloride ion as well. In one embodiment, the shielding element 3 is a curved thin structure having an elastic flexible material (such as plastic). In a cross-sectional view, an outer edge of the shielding element 3 is extended slightly inward to form a C-shape, so that the liquid absorbing and releasing element 4 will not fall off in a normal movement during the operation, but the liquid absorbing and releasing element 4 may be pulled out by a tool (such as tweezers) to facilitate cleaning or replacement. Since the liquid absorbing and releasing element 4 is tightly attached to the shielding element, the liquid absorbing and releasing element 4 will move synchronously with the shielding element 3 when the shielding element 3 moves above the silver layer 2 and the silver compound layer 2′(hereinafter the silver layer 2 and the silver compound layer 2′ are referred to as “electrode”), so that the electrochemical solution such as the potassium chloride solution (hereinafter, the potassium chloride solution represents the electrochemical solution) is coated uniformly onto the electrode. The shielding element 3 is deformed slightly when it is brushed or pushed by a user, so that the liquid absorbing and releasing element 4 in the shielding element 3 squeezes a small amount of potassium chloride solution to the electrode's surface. To absorb more potassium chloride solution onto the electrode surface, the electrode surface may be roughened by sandblasting, or the electrode surface may be scratched by a tool to form groove marks to accommodate more solution. On the other hand, gel or starch may be added to the potassium chloride solution to improve the viscosity of the solution, so that more solution stays on the electrode. Once the skin touches the electrode, the skin is further moistened, and an electrophysiological signal with better quality is obtained.

Please refer to FIG. 4B for a top view of a moving mechanism 6 and a shielding element in accordance with one embodiment of the present invention, the moving mechanism 6 may be a pivot, and 5 represents the element being situated in a position that covers the electrode, which is the original position before it moves. During use, a transversal force is applied slightly to push the shielding element, so that the shielding element is pushed away from the original position. After the shielding element rotates about the pivot 6 (pivot 6 is used as a rotation axis) by 90 degrees, it reaches-a position 7 as indicated by the arrow in the figure and exposes the electrode. Depending on the needs of the situation, the shielding element may be selectively coupled to a spring 11, so that after the pushing force is eliminated, the shielding element is pulled back to its original position to cover the electrode.

Please refer to FIG. 4C for a top view of moving mechanisms 10-1 and 10-2 and a shielding element 8 in accordance with one embodiment of the present invention, the moving mechanisms 10-1 and 10-2 form a set of rails. 8 represents the element being situated in a position that covers the electrode, which is the original position before it moves. After a transversal force is applied slightly to push the shielding element, the shielding element is pushed away from the original position to move horizontally along the rails 10-1 and 10-2 to position 9 as indicated by the arrow in the figure and exposes the electrode. Depending on the needs of the situation, the shielding element may be selectively coupled to a spring 12, so that after the pushing force is eliminated, the shielding element 5 is pulled back to its original position to cover the electrode.

According to a further embodiment of the present invention, the two kinds of moving mechanisms as shown in FIGS. 4B and 4C respectively may be selectively operated together with a fastener such as a latch as shown in FIG. 4F to form the embodiments as shown in FIGS. 4D and 4E, allowing the shielding element to rapidly leave the original position and expose the electrode. Please refer to FIGS. 4D and 4E, and FIG. 4F, FIG. 4F is a schematic view showing a fastener in accordance with one embodiment of the present invention, and the embodiments as shown in FIGS. 4D and 4E are substantially the same as the embodiment as shown in FIGS. 4B and 4C, except that the spring 11/12′ of the embodiment as shown in FIGS. 4D and 4E has an action force in a direction opposite to that of the embodiment as shown in FIGS. 4B and 4C. In other words, when the shielding element 5/8 is situated at the original position, the spring 11/12′ is compressed to store elastic potential energy, and the shielding element 5/8 is engaged with the fastener 14/15 and cannot be popped up. When it is necessary to record an electrocardiogram, a user gently presses a bump 17 of the locking member in the fastener 14/15 (as shown in FIG. 4F) to release its engagement with an opening of the shielding element 5/8, so as to drive the spring to release the elastic potential energy and push away the shielding element 5/8 quickly to expose the electrode. After the electrocardiogram is recorded, the user may apply a slight force to push the shielding element 5/8 from a position 7/9 back to the original position, and the bump 17 of the locking member is pushed upward by a cantilever 18 and engaged with the opening of the shielding element 5/8, so that the shielding element 5/8 is fixed to the original position and will not be pushed away by the spring. The advantage for the spring to push away the shielding element 5/8 quickly resides on that the user may capture the electrocardiogram within the shortest time which is very beneficial to an patient with early-stage heart disease (such as an occasional arrhythmia). The shielding element 5/8 has the function of preventing discomfort or injury caused by contacting the silver, chlorine, and potassium ions with the user's skin for a long time and preventing silver chloride from being rubbed and worn out. Therefore, when the electrocardiogram is not captured, the shielding element is situated at its original position, covering the electrode and preventing the electrode from touching the user's skin.

Only when it is necessary to capture an electrocardiogram, the shielding element leaves its original position to expose the electrode and allow the electrode to touch the user's skin.

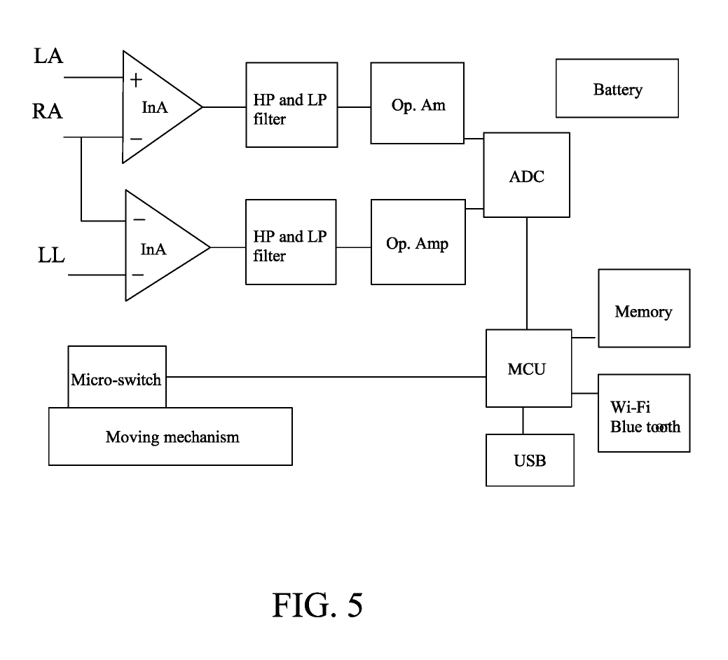

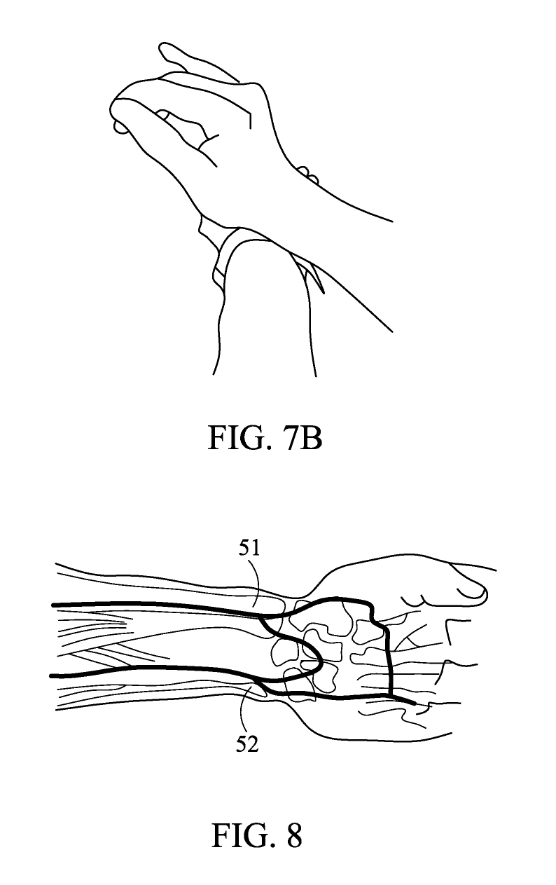

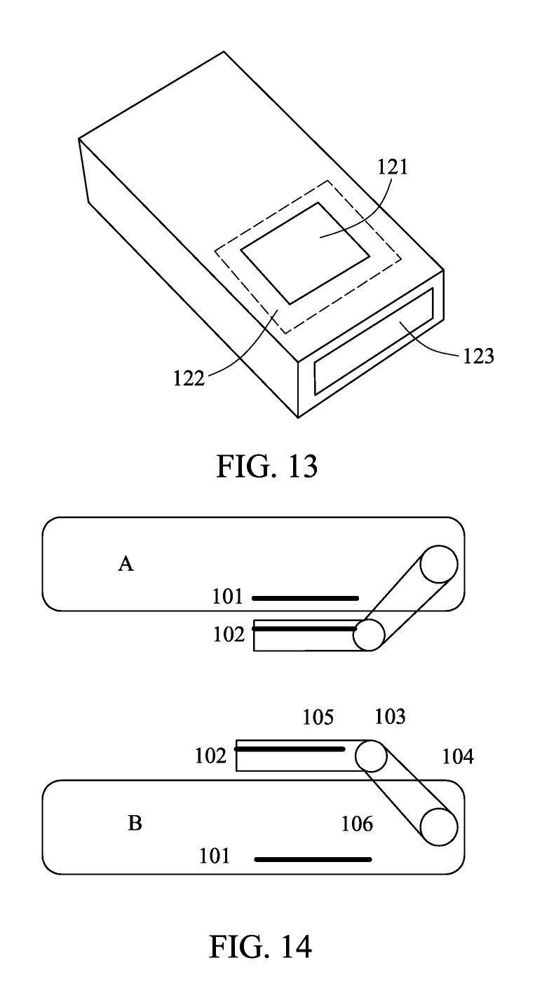

Please refer to FIG. 5 for an electrode device of the foregoing embodiment applied to the system structure of a wearable or handheld electrocardiogram recorder, LA represents an electrode in contact with the user's left hand, and RA represents an electrode in contact with the user's right hand, and LL represents an electrode in contact with the user's left leg, and the aforementioned three electrodes are respectively and electrically coupled to a positive input terminal and a negative input terminal of an instrumentation amplifier (InA) in order to eliminate common mode noises (mainly 60 Hz by AC power line). The output of the instrumentation amplifier goes through high-pass and low-pass filters (HP and LP filters) to stop low frequency noises (including drift by temperature change, electrochemical potential drift caused by ion diffusion, or drift caused by breathing or other muscle movements) and high frequency noises (including electromyogram, 60 Hz, radio wave, etc.), and then an operational amplifier (Op. Amp) amplifies the voltage, and an analog-digital converter (ADC) converts the analog signal into digital signal, and a microcontroller unit (MCU) processes the digital signal and stores the signal in a memory, and/or transmits the signal to other information communication apparatuses through a cable interface such as universal serial bus (USB), or a wireless interface such as Wi-Fi or Bluetooth. The circuit module described above is powered by a battery, and the microcontroller can turn on or off the power supply for aforementioned circuit modules. In addition, a micro switch (μ-switch) may be added and coupled to the microcontroller to show different logic circuit states when the shielding element is pushed away from the original position and returned to its original position respectively. By using the micro switch, the microcontroller may turn off each of the aforementioned circuit modules when the shielding element is situated at its original position, and the microcontroller also enters into a sleep mode. When the shielding element is pushed away, the electrocardiogram circuit is turned on, and such arrangement achieves the effects of saving power and extending the operating time. The system structure of the aforementioned electrocardiogram recorder may be implemented in a wearable apparatus such as a wrist watch body (as indicated by Element 87 of FIG. 7A), or implemented in a mobile phone (as shown in FIG. 13).

The electrode device of this embodiment may be installed to a wearable apparatus (such as a wrist watch) or a handheld apparatus (such as a mobile phone). When the electrode device is not in use for recording electrophysiological signals, the shielding element covers the electrode to prevent the electrode from touching a user's body, while preventing the evaporation of the electrochemical solution. If a user wants to record physiological signals, the user may push away the shielding element to expose the electrode and allow the electrode to be touched by the user's body. In the meantime, the liquid absorbing and releasing element in the shielding element is compressed to squeeze out the electrochemical solution to be coated onto the electrode to form a functional electrical conductor, so that the physiological signals can be captured successfully.

The embodiment mentioned above is applicable for capturing electrocardiac signals to produce an electrocardiogram (ECG) and also applicable for an electromyogram (EMG) and an electroencephalogram (EEG).

### Second Embodiment

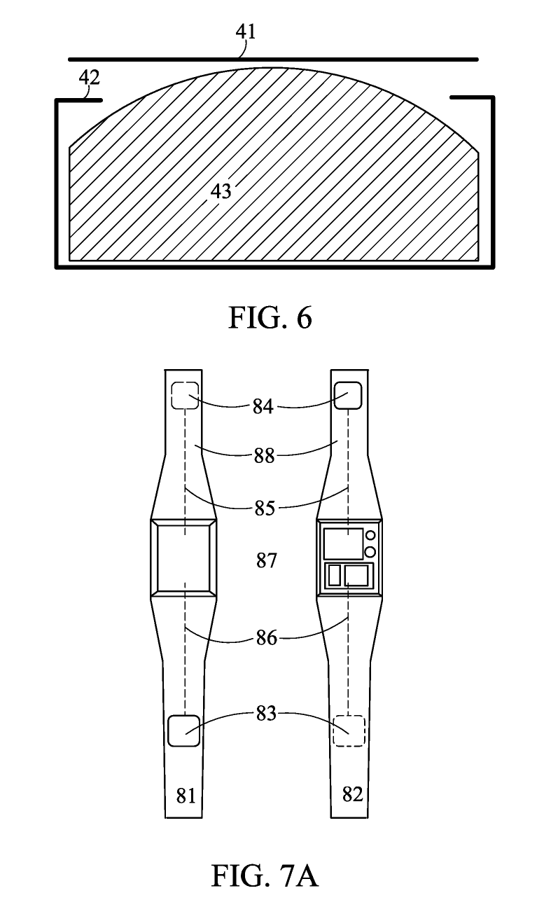

As for the electrode device installed on the inner side of a wrist watch, the shielding element of the electrode device of the first embodiment is too thick, making it difficult to put the shielding element onto the inner side of the wrist watch while allowing the shielding element to move, so the resistance is too large and causes discomfort. Please refer to FIG. 6 for a cross-sectional view of an electrode device in accordance with another embodiment of the present invention. As for an electrode device installed on the inner side of a wrist watch, FIG. 6 displays a more ideal electrode device, comprising a box-shaped silver layer and a box-shaped silver compound layer 42 used as electrodes (hereinafter referred to as “electrodes 42”) respectively, a liquid absorbing and releasing element 43 accommodated in the box-shaped electrode 42, a shielding element 41 covering the top of the electrode 42 and the liquid absorbing and releasing element 43, and a moving mechanism (not shown in FIG. 6, and its detail is shown in FIGS. 4B-4F) configured to move the shielding element 41 back and forth between at least two positions. This embodiment omits the conductive body. Of course, the electrode device of this embodiment may comprise a conductive body that covers the outer surface of, or is disposed below the electrode 42. Similar to the shielding element 3 of the first embodiment, an upper edge of the electrode 42 is extended inwardly to form C-shape for grasping and holding the liquid absorbing and releasing element 43, allowing the liquid absorbing and releasing element 43 to move with the electrode 42 in an operation and preventing liquid absorbing and releasing element 43 from falling off. After the liquid absorbing and releasing element 43 absorbs an aqueous electrochemical solution such as potassium chloride solution (For simplicity, the potassium chloride solution hereinafter represents the electrochemical solution), the liquid absorbing and releasing element 43 is slightly expanded, so that after the shielding element 41 is moved away, the liquid absorbing and releasing element 43 will be higher than the uppermost surface of the electrode 42. Then, it is compressed by the user's skin to release a small amount of potassium chloride solution to be coated onto the user's skin. Therefore, the electrophysiological signal can be conducted to the electrode 42. The shielding element 41 of this embodiment may have a porous design and a distance between the shielding element 41 and the electrode 42 be maintained, such that when the electrode device is not in use for measurement, so as to prevent the potassium chloride solution from touching the user's skin. During a measurement when the electrode 42 is pressed at a measuring point, the shielding element 41 may be moved downward while squeezing out the potassium chloride solution to touch the user's skin.

The electrode device of this embodiment may be applied to a wearable apparatus (such as a wrist watch). The application is described in details below. Please refer to FIG. 7A for a top view showing an electrode device in accordance with one embodiment of the present invention and the inner and outer sides of a wrist watch including the electrode device, the wrist watch includes an inner side 81 attached closely to a user's skin, and an outer side 82 situated opposite to the inner side 81, and comprises a watch body 87 comprising an electrocardiogram recorder as shown in FIG. 5, a band 88 configured to fix the watch body to the user's wrist, an inner electrode device 83 situated on the inner side, an outer side electrode device 84 situated on the outer side, and two connecting lines 85, 86 configured to connect the inner electrode device 83 and the outer side electrode device 84 respectively. The cross-sectional structure of the inner electrode device 83 is shown in FIG. 4 or FIG. 6.

Please refer to FIG. 8 for a schematic view of the inner side of the wrist of a human body. Wrist radial artery 51 and a wrist ulnar artery 52 are location on the inner side of the wrist, between the palm and forearm. Since there is no thick muscle between the epidermis and blood vessels, and only ligaments are attached to carpal ends, the structure is thinner than the structures at other positions, allowing it to have relatively lower resistance and not be affected by motion artifact caused by the contraction and extension of muscles. Therefore, such a position is most advantageous for conducting electrocardiogram signals. In addition, the inner side of the wrist is flat, allowing it to accommodate a large-area electrode and touch the user's skin with greater ease and stability. Therefore, a more ideal position of the inner electrode device 83 as shown in FIG. 7A is located in the middle of the inner side of the wrist (from the radial artery to the ulnar artery) as shown in FIG. 8.

In the same position of the inner electrode device 83, outer electrode device 84 is located on the outer surface of the band, it's cross-sectional structure is shown in FIG. 4 or FIG. 6 displaying a state of preparation for touching the epidermis at the positions from the radial artery 51 to the ulnar artery 52 of the wrist of the other hand. When the electrode device is not in use for electrocardiogram recording, the silver layer/silver compound layers (or electrodes) of both electrode devices 83, 84 are covered by the shielding element to prevent the electrodes from touching the user's skin. When the shielding element is pushed away, the user's skin touches the electrode, so that an electrocardiogram signal is conducted to an electrophysiological signal amplifier circuit, and then a microcontroller and a firmware process the signal to form an electrocardiogram, and a built-in wireless transmission device such as a Wi-Fi, Bluetooth, RFID, or infrared device of the wrist watch transmits the signal to another communication apparatus (such as a mobile phone or a personal computer) for display, storage, analysis, and diagnosis. Please refer to FIG. 7B for the status of a wrist watch when an electrocardiogram is recorded, both wrists touch the electrodes situated on the inner and outer sides of the wrist watch respectively to obtain a first lead (Limb Lead I) electrocardiogram.

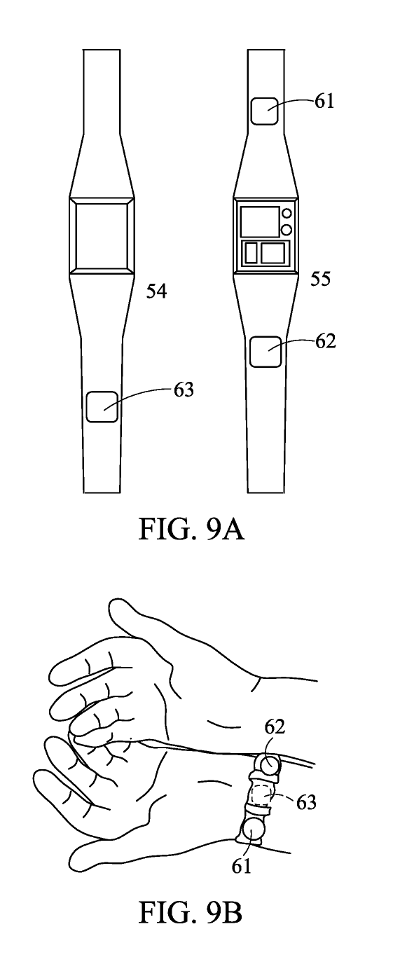

This embodiment of the present invention may be applied to obtaining the first, second and third lead electrocardiograms simultaneously. Please refer to FIG. 9A for a top view showing the inner and outer sides of a wrist watch in accordance with another embodiment of the present invention containing an electrode device of the present invention. FIG. 9B displays the status of the wrist watch depicted in FIG. 9A worn on the user's left hand. The wrist watch shown in FIG. 9A comprises an inner side 54 configured to attach closely to the user's skin and an outer side 55 situated opposite to the inner side 54, a watch body (not labeled), a band (not labeled), an inner electrode device 63 situated on the inner side 54, a first outer electrode device 61, and a second outer electrode device 62. Please refer to FIG. 4 or FIG. 6 for the cross-sectional structure of the inner electrode device 63. A preferred position of the inner electrode device 63 is between the radial artery 51 and the ulnar artery 52. Please refer to FIG. 4 or FIG. 6 for the cross-sectional structures of the first outer electrode device 61 and the second outer electrode device 62 respectively; their preferred positions are in the middle between the radial artery 51 and the ulnar artery 52.

In FIG. 9B, when the left palm faces upward, the first outer electrode device 61 is disposed on a side closer to the user's left thumb, and the second outer electrode device 62 is disposed on the other side closer to the user's left little finger, and the inner electrode device 63 is disposed between the first outer electrode device 61 and the second outer electrode device 62 (wherein the dotted line in the FIG. 9B indicates the inner electrode device 63 being disposed on the inner side and covered by the band).

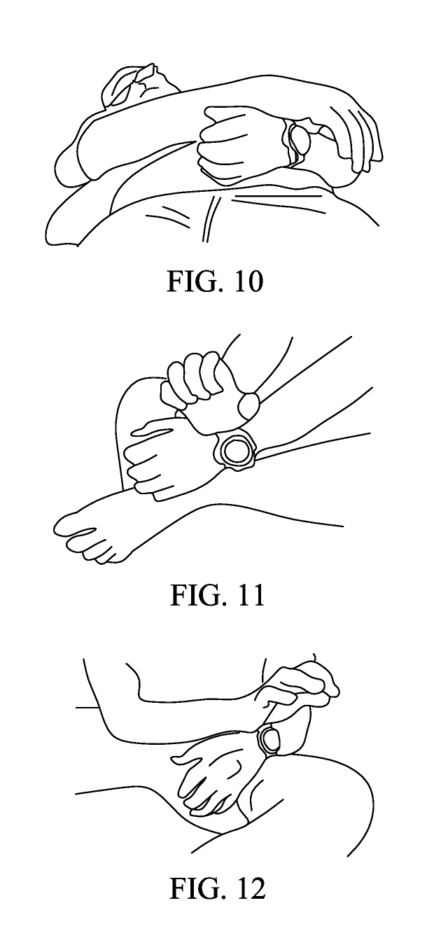

Please refer to FIGS. 10, 11 and 12 for different states of a wrist watch as depicted in FIG. 9A when the first, second and third lead electrocardiograms are recorded. FIG. 10 shows a user wearing the wrist watch depicted in FIG. 9A on the user's left hand to record the first, second and third lead electrocardiograms. In order to record electrocardiograms, the second outer electrode device 62 of the wrist watch touches close to a position below the user's heart (which is the user's left abdomen), and the left hand applies a transversal force toward the left, moving away the shielding element to allow the three electrode devices to touch the user's left hand, right hand, and left abdomen respectively to obtain the first, second and third lead electrocardiograms simultaneously. If it is inconvenient for the user to touch the left abdomen with the wrist watch due to the limitation of clothing, the user may use the wrist watch to a part of the user's body below the left side of the user's heart such as the left ankle or left thigh as shown in FIG. 11 or FIG. 12, so as to obtain the first, second and third lead electrocardiograms simultaneously.

In the embodiment described above, the electrode device disposed on the outer side preferably has a cross-sectional structure as shown in FIG. 4, and the electrode device disposed on the inner side preferably has a cross-sectional structure as shown in FIG. 6. To guide users to operate the electrode device correctly, the wrist watch body may comprise several different colored LED lamps, a buzzer, LCD or a voice generator for prompting the user when R peaks are detected.

Compared with conventional techniques, this embodiment has a silver layer/silver chloride layer/potassium chloride solution to enhance the electrical conduction, and a lower electrical impedance occurs between a portion of a human body in contact with the electrode device and large blood vessels, so that the electrocardiograph amplifier circuit has a shorter transient time (less than 0.5 second), and an effective electrocardiogram may be obtained in a shorter time which is advantageous to the patients with early-stage heart disease. In addition, there is no muscle between the electrode device and the large blood vessels, and no motion artifact is formed due to muscle contraction and extension, so that an electrocardiogram with better quality is obtained.

### Third Embodiment

The following limitation must be taken into consideration when a handheld apparatus such as a mobile phone is used to capture electrophysiological signals. In general, most mobile phones come with a touch panel on the front side and a layer of insulator as the surface in order to detect the capacitance varied with the force applied on the touch panel by fingers. Please refer to FIG. 13 for side views of a mobile phone including an electrode device of the present invention in accordance with one embodiment, A and B indicates the statuses of not capturing and capturing an electrophysiological signal respectively. To use the mobile phone to capture an electrophysiological signal, the second embodiment comprises two electrode devices installed on a back side of the mobile phone, and the mobile phone includes an apparatus body (not labeled but indicated by a large rectangle), rotating arms 105, 106, of pivots 103, 104, a first electrode device 101 fixed to the back side of the mobile phone, and a second electrode device 102 coupled to the rotating arm 105 which have the function of a shielding element. The rotating arms 105, 106 are coupled to each other by the pivot 103, and further coupled to the mobile phone body by the pivot 104. Since statuses A and B involve the same elements, some of the numerals in status A are omitted for simplicity.

As shown in status A, when the electrophysiological signal is not captured, the second electrode device 102 is situated at the back side of the mobile phone to cover the first electrode device 101. In status B, when it is necessary to capture an electrophysiological signal, a user turns the second electrode device 102 to the front side of the mobile phone front side by using the rotating arms 105, 106 and the pivots 103, 104, so that both of the front and back sides of the mobile phone have an electrode device, and the user may touch both sides of the mobile phone with two wrists respectively to obtain the electrophysiological signal. To fix the rotating arms into a stable position, either a permanent magnet is installed on the rotating arm and the mobile phone body, or a permanent magnet is installed at one end while a plate made of a magnetic material (such as an iron plate) is installed at the other end, so that the rotating arm has an appropriate adhesion, requiring the user to apply a pulling force to detach the rotating arm from the mobile phone body. In another mode, a spring may be added to the position of the pivot to achieve the same effect.

Similar to the advantages of the second embodiment, compared with conventional techniques, this embodiment obtains an effective electrocardiogram in a shorter amount of time, and the electrode device is attached to a particular part of the user's body to avoid thick muscles and tissues, reducing the signal motion artifact caused by muscle contraction and extension so that an electrocardiogram with better quality is obtained in such measurement.

### Fourth Embodiment

Please refer to FIG. 14 for a modification of one embodiment in FIG. 13. FIG. 14 shows a handheld apparatus such as a mobile phone in accordance with the third embodiment having a third electrode device 123 of the first embodiment added onto one of the mobile phone's lateral sides. Therefore, the handheld apparatus as shown in FIG. 14 has three electrode devices: the movable second electrode device 121 of the third embodiment, the first electrode device 122 installed onto the back side of the handheld apparatus of the third embodiment, and the third electrode device 123 added to the lateral side of the handheld apparatus. The first, second, and third electrode devices are respectively and electrically coupled to first, second, and third input terminals of an electrocardiograph amplifier circuit of a main body of the handheld apparatus to record the three limb lead (Limb Leads I, II, III) electrocardiograms defined by Einthoven. When it is necessary to record an electrocardiogram, the user uses the inner sides of both wrists to touch the front and back sides of the handheld apparatus respectively to clamp the handheld apparatus. Similar to the second embodiment, the lateral side of the handheld apparatus touches the skin of the user's left lower abdomen or left leg in order to capture the electrocardiogram.

Compared with conventional techniques, this embodiment has a silver layer/silver chloride layer/potassium chloride solution to assist electrical conduction, and a lower electrical impedance occurs between a portion of a human body in contact with the electrode device and large blood vessels, so that the electrocardiograph amplifier circuit has a shorter transient time (less than 0.5 second), and an effective electrocardiogram may be obtained in a shorter time which is advantageous to the patients with early-stage heart disease. As to patients having a sudden heart attack, the apparatus may be operated by the patient or by other people to obtain a clear electrocardiogram quickly, which allows the results to be sent immediately to family, friends and medical care units, so that the patient can be treated and taken care of as soon as possible.

### Fifth Embodiment

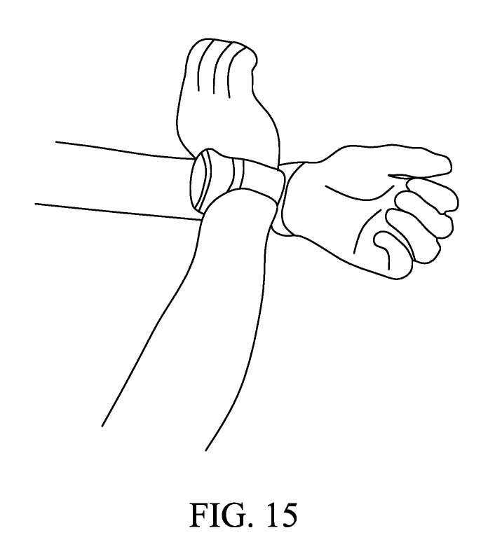

Please refer to FIG. 15 for the status of a wearable apparatus worn on both wrists in accordance with one embodiment of the present invention including the electrode device of the present invention. The wearable apparatus has a primary wristband and a secondary wristband worn on both wrists as shown in FIG. 15, and the two wristbands include the electrode devices of the first embodiment. In FIG. 15, the primary wristband and the secondary wristband may be worn on the user's left and right wrists or the user's right and left wrist respectively according to the user's preference, so that the user can obtain the electrocardiogram easily and quickly.

The primary wristband includes a main body, a band, an electrode device, a conductive contact and two connecting chords. The secondary wristband is simpler than the primary wristband, including just a band, a conductive contact, an electrode device and a connecting chord. The main body includes an electrocardiograph amplifier circuit, a microcontroller, a battery, memory and a control firmware. In the main body, the battery is heavier than the other parts. For comfort and convenience, the preferred position of installing the electrode device is on the back of the hand (the position where people generally wear watches), allowing space for the electrode device to come into contact with the user's skin at the position between the radial and ulnar arteries.

The electrode devices of both primary and secondary wristbands are installed on the inner side of the band and configured to situate between the radial and ulnar arteries of the wrist. As the first embodiment shown in FIG. 4A, the electrode device comprises a silver layer/silver compound layer, a shielding element, a liquid absorbing and releasing element, and a moving mechanism.

In the primary wristband, a first connecting chord is configured to connect the electrode device to a first input terminal of the electrocardiograph amplifier circuit, and a second connecting chord is configured to connect the conductive contact to a second input terminal of the electrocardiograph amplifier circuit. In the secondary wristband, a connecting chord is configured to connect an electrode device of the secondary wristband to a conductive contact.

When it is necessary to record an electrocardiogram signal, a user moves both arms to touch the conductive contacts of the two wristbands. Similar to the first embodiment, a slight force is applied to move away the shielding elements of the primary wristband and the secondary wristband to expose the electrodes, so that the electrodes can conduct the electrocardiogram signal of the wrist through the connecting line and the conductive contact to the amplifier input terminal of the main body, so as to obtain the Limb Lead I electrocardiogram.

Similar to the second embodiment, a micro switch may be added to the primary wristband and coupled to the microcontroller, such that different logic circuit states are shown when the shielding element is pushed away or restored to its original position, so as to save power consumption. Alternatively, two conductive contacts are installed next to the conductive contact on the outer side of the primary wristband and connected to the microcontroller respectively, and a conductor is installed at a corresponding position of the secondary wristband. When the primary wristband and the secondary wristband come into contact with each other, the two contacts also touch the conductor for electrical conduction, allowing the microcontroller to determine whether or not the primary wristband and the secondary wristband are connected. Similarly, the aforementioned two contacts may be substituted by two reed switches, and the aforementioned conductor may be substituted by a permanent magnet to achieve the same effect.

Although the invention has been described with reference to specific embodiments, this description is not meant to be construed in a limiting sense. Various modifications of the disclosed embodiments as well as alternative embodiments of the invention will become apparent to persons skilled in the art. It is therefore contemplated that the appended claims will cover any such modifications or embodiments that fall within the scope of the invention.

What is claimed is:

1. An electrode device for capturing an electrophysiological signal comprising:
a conductive body;
a silver layer situated above the conductive body;
a silver compound layer situated above the silver layer;
a liquid absorbing and releasing element situated above the silver compound layer;
a shielding element covering an upper surface and a portion of a side surface of the liquid absorbing and releasing element; and
a moving mechanism configured to move the shielding element back and forth between at least two positions.

2. The electrode device for capturing an electrophysiological signal according to claim 1, wherein the shielding element is configured to couple a spring.

3. The electrode device for capturing an electrophysiological signal according to claim 1, wherein the shielding element is configured to engage a fastener.

4. The electrode device for capturing an electrophysiological signal according to claim 1, wherein the shielding element is configured to couple a micro switch.

5. The electrode device for capturing an electrophysiological signal according to claim 1, wherein the shielding element has a curved thin structure having an elastic flexible material.

6. The electrode device for capturing an electrophysiological signal according to claim 1, wherein the shielding element has an inwardly extended edge configured to grip the liquid absorbing and releasing element.

7. The electrode device for capturing an electrophysiological signal according to claim 1, wherein the silver layer and the silver compound layer jointly have an inwardly extended edge configured to grip the liquid absorbing and releasing element.

8. A wearable apparatus for electrocardiogram recording comprising:
an inner side of the wearable apparatus and an outer side of the wearable apparatus opposite to the inner side of the wearable apparatus;
a main body including an electrocardiograph amplifier circuit, a microcontroller, a memory, a battery and a control firmware;
a first electrode and a second electrode, each of the first electrode and the second electrode including a silver layer, a silver compound layer, a shielding element, a liquid absorbing and releasing element, and a moving mechanism,
wherein the first electrode is disposed on the inner side of the wearable apparatus and configured to situate between radial and ulnar arteries of a wrist, and the second electrode is disposed on the outer side of the wearable apparatus,
wherein the first electrode and the second electrode are connected to two input terminals of the electrocardiograph amplifier circuit respectively.

9. The wearable apparatus for electrocardiogram recording according to claim 8, further comprising a third electrode device disposed on the outer side of the wearable apparatus, wherein the third electrode device includes a silver layer, a silver compound layer, a shielding element, a liquid absorbing and releasing element, and a moving mechanism.

10. The wearable apparatus for electrocardiogram recording according to claim 9, wherein the third electrode device is configured to couple to a third input terminal of the electrocardiograph amplifier circuit.

11. The wearable apparatus for electrocardiogram recording according to claim 8, wherein the wearable apparatus is a wrist watch.

12. The wearable apparatus for electrocardiogram recording according to claim 8, further comprising a built-in wireless transmission device configured to transmit the electrocardiogram to another communication apparatus for display, storage, analysis and/or diagnosis.

13. A handheld apparatus for electrocardiogram recording comprising:
a body having a first side and a second side opposite to the first side, wherein the body further includes an electrocardiograph amplifier circuit, a microcontroller, a wireless transmission circuit, a memory, a battery, and a control firmware; and
a first electrode device and a second electrode device, the first electrode device being situated on the first side of the body and including a silver layer, a silver compound layer, and a liquid absorbing and releasing element, and the second electrode device being connected to the body by a rotating arm and a pivot, wherein the first electrode device and the second electrode device are electrically coupled to two input terminals of the electrocardiograph amplifier circuit respectively,
wherein when the handheld apparatus is in a non-recording status, the second electrode device is configured to cover the first electrode device to prevent the first electrode device from being exposed, and when the handheld apparatus is in a recording status, the second electrode is configured to be turned to the second side of the body to expose the first electrode device and the second electrode device to be touched by a user.

14. The handheld apparatus for electrocardiogram recording according to claim 13, further comprising:
a permanent magnet configured to make the second electrode device abut against the first electrode device.

15. The handheld apparatus for electrocardiogram recording according to claim 13, further comprising:
a spring configured to make the second electrode device abut against the first electrode device.

16. The handheld apparatus for electrocardiogram recording according to claim 13, further comprising:
a third electrode device situated on a lateral side of the handheld apparatus and including a silver layer, a silver compound layer, a shielding element, a liquid absorbing and releasing element, and a moving mechanism, wherein the third electrode device is electrically coupled to a third input terminal of the electrocardiograph amplifier circuit.

17. A wrist-worn apparatus for electrocardiogram recording comprising:
a primary wristband including a main body, a first band, a first electrode device, a first conductive contact, and two first connecting chords, the main body including an electrocardiograph amplifier circuit, a microcontroller, a battery, a memory, and a control firmware,
wherein the first electrode device is disposed on an inner side of the first band and is configured to situate between radial and ulnar arteries of one wrist of a user,
wherein the first electrode device includes a first silver layer, a first silver compound layer, a first shielding element, a first liquid absorbing and releasing element, and a first moving mechanism,
wherein the first electrode device is connected to a first input terminal of the electrocardiograph amplifier circuit by one of the two first connecting chords, and the first conductive contact is connected to a second input terminal of the electrocardiograph amplifier circuit by the other one of the two first connecting chords; and
a secondary wristband including a second band, a second conductive contact, a second electrode device and a second connecting chord,
wherein the second electrode device is disposed on an inner side of the second band and is configured to situate between radial and ulnar arteries of the other wrist of the user,
wherein the second electrode device includes a second silver layer, a second silver compound layer, a second shielding element, a second liquid absorbing and releasing element, and a second moving mechanism,
wherein the second electrode device is connected to the second conductive contact by the second connecting chord,
wherein the primary wristband and the secondary wristband are configured to be worn on the one wrist and the other wrist of the user respectively.

18. The wrist-worn apparatus for electrocardiogram recording according to claim 17, wherein the primary wristband further comprises a switch configured to show different logic states to the microcontroller with one of the different logic states indicating the primary wristband and the secondary wristband being in contact while the other of the different logic states indicating the primary wristband and the secondary wristband being not in contact.
